(19) United States
(12) Patent Application Publication
(10) Pub. No.: US 2005/0058600 A1
(43) Pub. Date: Mar. 17, 2005

(54) 3,3'-Diindolylmethane antiandrogenic compositions

(76) Inventors: Leonard F. Bjeldanes, Berkeley, CA (US); Hien T. Le, Berkeley, CA (US); Gary L. Firestone, Berkeley, CA (US)

Correspondence Address: RICHARD ARON OSMAN;SCIENCE AND TECHNOLOGY LAW GROUP, 242 AVE VISTA DEL OCEANO, SAN CLEMEMTE, CA, 92672, US

(21) Appl. No.: 10/664,991

(22) Filed: Sep. 16, 2003

(51) Int. Cl. A61K 49/00; A61K 31/138

(52) U.S. Cl. 424/9.1; 514/651

(57) ABSTRACT

The invention provides antiandrogenic compositions and methods of use. The general methods deliver an antiandrogen to a host determined to be in need thereof by contacting the host with an effective amount of an antiandrogenic, optionally substituted 3,3′-diindolylmethane (DIM); and detecting a resultant antiandrogenic response in the host. The method may further comprise, prior to the contacting step, determining that the host is in need of the antiandrogen.

This work was supported by National Institute of Environmental Health Sciences Center Grant P30ESO1896. The U.S. government may have rights in any patent issuing on this application.

### Introduction

### Field of the Invention

The field of the invention is 3,3′-diindolylmethane compositions and their use as antiandrogens.

### Background of the Invention

Prostate cancer is the second leading cause of cancer related mortalities in American men with more than 40,000 deaths in 1997 (1). One of every four cancers diagnosed is of prostatic origin, making prostate cancer the most commonly diagnosed cancer (2). Although the incidence of prostate cancer in Japanese and Chinese men is remarkably low compared to the incidence in American males, following migration to the US, the risk of later generations of Asian immigrants rises to levels that are similar to American males (3, 4). The differences in prostate cancer diagnosed among various population groups suggest that factors in the environment, lifestyles, and diet play a role in prostate cancer initiation and/or progression.

One possible contributor to the lower prostate cancer rates in Asian men is the higher consumption of phytochemical-rich vegetables that is typical of this population (5, 6). Consumption of cruciferous vegetables, including broccoli, Brussels sprouts, kale, and cauliflower has been associated with decreased risk of various human cancers. The strongest associations are with cancers of the breast, endometrium, colon, and prostate (7-10).

Incorporation of Brassica plants in feed reduces spontaneous and carcinogen induced tumorigenesis in experimental animals, with the greatest protective effects seen in mammary tumors (11-13). A major active compound in cruciferous vegetables, indole-3-carbinol (I3C), along with its digestive derivative, 3,3′-diindolylmethane (DIM), exhibit promising cancer protective properties in vivo and in vitro. These compounds reduced the incidence of dimethylbenzanthracene (DMBA)-induced mammary tumors in rats, benzo(a)pyrene (BP)-induced tumors of the forestomach in mice, and BP-induced pulmonary adenomas in mice (14, 15). I3C has been shown to inhibit proliferation of both breast (16, 17) and prostate cancer cells (18, 19) by blocking the cell cycle and inducing apoptosis. DIM has been reported to inhibit proliferation and induced programmed cell death in human breast tumor cells in culture (20, 21).

The cancer preventive effects of DIM, especially on hormone-mediated breast cancer led us to investigate the effects and mechanism of action of DIM against proliferation of other tumor cells. In the course of these studies, we made the surprising discovery that DIM is a potent antiandrogen. We reveal here our structure-activity relationship (SAR) studies which show antiandrogenic effects of DIM and a variety of substituted 3,3′-diindolylmethane compounds. Our results establish DIM as a unique bifunctional hormone disrupter, and the first example of a pure androgen receptor antagonist from plants.

### SUMMARY OF THE INVENTION

The invention provides antiandrogenic compositions and methods of use. The general methods deliver an antiandrogen to a host determined to be in need thereof by contacting the host with an effective amount of an antiandrogenic, optionally substituted 3,3′-diindolylmethane (DIM); and detecting a resultant antiandrogenic response in the host. The method may further comprise, prior to the contacting step, determining that the host is in need of the antiandrogen. In a preferred embodiment, the host is a human patient determined to be subject or predisposed to an androgen-dependent pathology, and the resultant antiandrogenic response is a reduction in the pathology or progress of the pathology, particularly a pathology selected from the group consisting of prostate hyperplasia, acne, androgenic alopecia and hirsutism, and the resultant antiandrogenic response is a reduction in the pathology or progress of the pathology.

The methods generally employ an antiandrogenic, optionally substituted 3,3′-diindolylmethane having formula I: 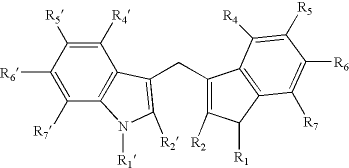
where R.sub.1, R.sub.2, R.sub.4, R.sub.5, R.sub.6, R.sub.7, R.sub.1′, R.sub.2′, R.sub.4′, R.sub.5′, R.sub.6′ and R.sub.7′ individually and independently, are hydrogen or a substituent selected from the group consisting of a halogen, a hydroxyl, a linear or branched alkyl or alkoxy group of one to ten carbons, an amine, a sulfonyl, and a nitro group. In particular embodiments, the compound includes at least one such substituent, preferably at a position other than, or in addition to R.sub 1 and R.sub 1′, the linear or branched alkyl or alkoxy group is one to five carbons, and/or the halogen is selected from the group consisting of chlorine, bromine and fluorine. In particular embodiments, the indolyls are symmetrically substituted, wherein each indolyl is similarly mono-, di-, tri-, or para-substituted.

The invention also provides methods of using an antiandrogenic, optionally substituted 3,3′-diindolylmethane in conjunction with one or more other therapeutic agents, particularly different antiandrogenic compounds, for complementary, additive, and/or synergistic efficacy. These methods may employ combination compositions, which may be in combination unit dosages, or separate compositions, which may be provided separately dosed in joint packaging.

The invention also provides kits comprising an antiandrogenic, optionally substituted 3,3′-diindolylmethane, and an instructional medium reciting a subject method. The recited antiandrogenic, optionally substituted 3,3′-diindolylmethane may be present in premeasured, unit dosage, and may be combined in dosage or packaging with an additional therapeutic agent, particularly a different antiandrogen.

### DETAILED DESCRIPTION OF PARTICULAR EMBODIMENTS OF THE INVENTION

The following descriptions of particular embodiments and examples are offered by way of illustration and not by way of limitation. Unless contraindicated or noted otherwise, in these descriptions and throughout this specification, the terms “a” and “an” mean one or more, the term “or” means and/or.

The general methods deliver an antiandrogen to a host determined to be in need thereof by contacting the host with an effective amount of an antiandrogenic, optionally substituted 3,3′-diindolylmethane (DIM); and detecting a resultant antiandrogenic response in the host. The method may further comprise, prior to the contacting step, determining that the host is in need of the antiandrogen.

The disclosed antiandrogenic compositions provide diverse applications to hosts determined to be in need of an antiandrogen. For example, scientists can use the compositions to provide antiandrogenic activity to cell cultures or laboratory animals. Preferred hosts are human patients, wherein the compositions may be administered to patients determined to be subject or predisposed to any of the wide variety of disorders known to be treatable with antiandrogens. Examples include use as an antineoplastic agent and palliative, adjuvant or neoadjuvant hormonal therapy in prostate cancer, treatment of benign prostatic hyperplasia (prostate enlargement), acne, androgenetic alopecia (male pattern baldness), hirsutism (excessive hairiness), polycystic ovary syndrome (PCOS), menopausal discomfort, male contraception, to purposefully prevent or counteract masculinisation in the case of male to female transsexuals, and to prevent the symptoms associated with reduced testosterone, like hot flashes, following castration. The administration of the subject antiandrogens in males can result in slowed or halted development or reversal of male secondary sex characteristics, reduced activity or function of the accessory male sex organs, and hyposexuality (diminished sexual desire or libido). Hence, the compositions may also be used in severe male sexual disorders, such as hypersexuality (excessive sexual desire) and sexual deviation, specifically paraphilias.

In a preferred embodiment, the host is a human patient determined to be subject or predisposed to an androgen-dependent pathology, and the resultant antiandrogenic response is a reduction in the pathology or progress of the pathology, particularly a pathology selected from the group consisting of prostate hyperplasia, acne, androgenetic alopecia and hirsutism, and the resultant antiandrogenic response is a reduction in the pathology or progress of the pathology.

Our methods generally employ an antiandrogenic, optionally substituted 3,3′-diindolylmethane having the structure of formula I, wherein R.sub.1, R.sub.2, R.sub.4, R.sub.5, R.sub.6, R.sub.7, R.sub.1′, R.sub.2′, R.sub.4′, R.sub.5′, R.sub.6′ and R.sub.7′ individually and independently, are hydrogen or a substituent selected from the group consisting of a halogen, a hydroxyl, a linear or branched alkyl or alkoxy group of one to ten carbons, an amine, a sulfonyl, and a nitro group. Substituent-containing compounds may be referred to as DIM derivatives or DIM analogs. In particular embodiments, the compound includes at least one such substituent, preferably at a position other than, or in addition to R.sub 1 and R.sub 1′, the linear or branched alkyl or alkoxy group is one to five carbons, and/or the halogen is selected from the group consisting of chlorine, bromine and fluorine.

Antiandrogen activity is readily confirmed with the various assays described below, including androgen-dependent cell proliferation assays, androgen-dependent DNA synthesis and transcriptional reporter assays, PSA transcription and expression assays, androgen competitive binding and binding inhibition assays, etc. In addition, a wide variety of clinically relevant and validated animal models for antiandrogen activity are well-known in the art, e.g. Yamada et al., 2000, Toxicological Sciences 53, 289-296; O'Conner et al. Toxicological Sciences 69, 92-108 (2002).

In particular, we devised an iterative, combinatorial synthetic scheme to generate a library of DIM derivatives for high-throughput screening of antiandrogen activity. In an exemplary demonstration, we generated 451 DIM derivative structures in five synthetic rounds, summarized below:
Synthetic round 2: R sub.2, 4, 5, 6 or 7, R sub.2′, 4′, 5′, 6′ or 7′ di-F, -Cl, or -Br-3,3′, -diindolylmethane
Synthetic round 5: R sub.2, 4, 5, 6 or 7, R sub.2′, 4′, 5′, 6′ or 7′ di-methyl-, ethyl-, propyl butyl-, or pentyl-3,3′-diindolylmethane
Synthetic round 6: R sub.2, 4, 5, 6 or 7, R sub.2′, 4′, 5′, 6′ or 7′ di-methoxy-, ethoxy-, propyloxy-, butyloxy-, or pentyloxy-3,3′-diindolylmethane
Synthetic round 9: R sub.2, 4, 5, 6 or 7, R sub.2′, 4′, 5′, 6′ or 7′ di-hydroxyl, amino-, aminomethyl-, sulfo-, or nitro-3,3′-diindolylmethane
Synthetic round 12: R sub.2, 4, 5, 6, 7, R sub. 2′, 4′, 5′, 6′, 7′ deca-fluoro (perfluoro)-3,3′-diindolylmethane

The antiandrogenic effects of the DIM analogs are examined with a reporter assays using a MMTV-Luc promoter construct that contains one ARE and a pPSA-Luc promoter construct containing three AREs. These plasmids were transiently transfected into LNCaP cells, and by luciferase analysis, showed that DIM strongly inhibited DHT induction of androgen-responsive genes by over 50% at 1 μM, and over 90% at 10 μM in both promoter constructs. Treatment with DIM alone failed to induce transactivation of these reporter genes. Similar results with our combinatorial library identify antiandrogenic DIM analog inhibition of AR-responsive gene expression. DIM derivatives confirmed to have antiandrogenic activity in our reporter assay are subsequently screened in a whole animal Hersenberger assay, as described below in Example II.

In particular embodiments, the indolyl moieties are symmetrically substituted, wherein each moiety is similarly mono-, di-, tri-, etc. substituted. In other particular embodiments, R.sub.1, R.sub.2, R.sub.4, R.sub.6, R.sub.7, R.sub.1′, R.sub.2′, R.sub.4′, R.sub.6′, and R.sub.7′ are hydrogen, R.sub.5 and R.sub.5′ are a halogen selected from the group consisting of chlorine, bromine and fluorine. Additional DIM derivatives from which antiandrogenic compounds are identified as described herein include compounds wherein R.sub. 1, R.sub.2, R.sub.4, R.sub.6, R.sub.7, R.sub.1′, R.sub.2′, R.sub.4′, R.sub.6′, and R.sub.7′ are hydrogen, R.sub.5 and R.sub.5′ are halogen. These include, but are not limited to 3,3′-diindolylmethane, 5,5′-dichloro-diindolylmethane; 5,5′-dibromo-diindolylmethane; 5,5′-difluoro-diindolylmethane. Additional preferred such DIM derivatives include compounds wherein R.sub. 1, R.sub.2, R.sub.4, R.sub.6, R.sub.7, R.sub.1′, R.sub.2′, R.sub.4′, R.sub.6′, and R.sub.7′ are hydrogen, R.sub.5 and R.sub.5′ are an alkyl or alkoxyl having from one to ten carbons, and most preferably one to five carbons. These include, but are not limited to 5,5′-dimethyl-diindolylmethane, 5,5′-diethyl-diindolylmethane, 5,5′-dipropyl-diindolylmethane, 5,5′-dibutyl-diindolylmethane and 5,5′-dipentyl-diindolylmethane. These also include, but are not limited to, 5,5′-dimethoxy-diindolylmethane, 5,5′-diethoxy-diindolylmethane, 5,5′-dipropyloxy-diindolylmethane, 5,5′-dibutyloxy-diindolylmethane, and 5,5′-diamyloxy-diindolylmethane.

Additional preferred such DIM derivatives include compounds wherein R.sub.2, R.sub.4, R.sub.5, R.sub.6, R.sub.7, R.sub.2′, R.sub.4′, R.sub.5′, R.sub.6′, and R.sub.7′ are hydrogen, R.sub.1 and R.sub.1′ are an alkyl or alkoxyl having from one to ten carbons, and most preferably one to five carbons. Such useful derivatives include, but are not limited to, N,N′-dimethyl-diindolylmethane, N,N′-diethyl-diindolylmethane, N,N′-dipropyl-diindolylmethane, N,N′-dibutyl-diindolylmethane, and N,N′-dipentyl-diindolylmethane. In yet another preferred embodiment, R.sub.1, R.sub.4, R.sub.5, R.sub.6, R.sub.7, R.sub.1′, R.sub.4′, R.sub.5′, R.sub.6′, and R.sub.7′ are hydrogen R.sub.2 and R.sub.2′ are alkyl of one to ten carbons, and most preferably one to five carbons. Such compounds include, but are not limited to, 2,2′-dimethyl-diindolylmethane, 2,2′-diethyl-diindolylmethane, 2,2′-dipropyl-diindolylmethane, 2,2′-dibutyl-diindolylmethane, and 2,2′-dipentyl-diindolylmethane. In another embodiment, R.sub.1, R.sub.2, R.sub.4, R.sub.6, R.sub.7, R.sub.1′, R.sub.2′, R.sub.4′, R.sub.6′, and R.sub.7′ are hydrogen, R.sub.5 and R.sub.5′ are nitro.

Substituted DIM analogs are readily prepared by condensation of formaldehyde with commercially available substituted indoles. Precursor compounds can be synthesized by dimethylformamide condensation of a suitable substituted indole to form a substituted indole-3-aldehyde. Suitable substituted indoles include indoles having substituents at R.sub. 1, R.sub.2, R.sub.4, R.sub.5, R.sub.6 and R.sub.7 positions. These include, but are no limited to 5-methoxy, 5-chloro, 5-bromo, 5-fluoro, 5′-methyl, 5-nitro, n-methyl and 2-methyl indoles. The substituted indole 3-aldehyde product is treated with a suitable alcohol such as methanol and solid sodium borohydride to reduce the aldehyde moiety to give substituted I3Cs. Substituted DIMs are prepared by condensing the substituted indole-3-carbinol products. This may be achieved, for example, by treatment with a phosphate buffer having a pH of around 5.5.

The antiandrogens may be administered along with a pharmaceutical carrier and/or diluent. The antiandrogens of the present invention may also be administered in combination with other agents, for example, in association with other chemotherapeutic or immunostimulating drugs or therapeutic agents. Examples of pharmaceutical carriers or diluents useful in the present invention include any physiological buffered medium, i.e., about pH 7.0 to 7.4 comprising a suitable water soluble organic carrier. Suitable water-soluble organic carriers include, but are not limited to corn oil, dimethylsulfoxide, gelatin capsules, etc.

In particular embodiments, the invention provides methods of using the subject antiandrogenic, optionally substituted 3,3′-diindolylmethane in conjunction with one or more other therapeutic agents, particularly different antiandrogenic compounds, for complementary, additive, and/or synergistic efficacy. Exemplary, well-known antiandrogens useful in such combinations, include:
Bicalutamide (Casodex): a nonsteroidal antiandrogen, administered orally, typically used in combination with a luteinizing hormone-releasing hormone (LHRH) agonist in treatment of prostate cancer;
Cyproterone acetate (Androcur): a synthetic steroid, a potent antiandrogen that also possesses progestational properties;
Finasteride (Proscar, Propecia): a synthetic enzyme inhibitor, known as a 5-αa-reductase (type II) inhibitor, that prevents dihydrotestosterone synthesis, administered orally, mainly used for treatment of benign prostate enlargement and as a hair growth stimulant;
Dutasteride (Avodart): a 5αa-reductase inhibitor, that also blocks 5αa-reductase type I;
Flutamide (Eulexin): a nonsteroidal androgen receptor blocker often used to treat hirsutism and metastatic prostate cancer, and to increase the flow of urine in benign prostate enlargement;
Ketoconazole (Nizoral): an imidazole derivative used as a broad-spectrum antifungal agent effective against a variety of fungal infections, though side effects include serious liver damage and reduced levels of androgen from both the testicles and adrenal glands;
Nilutamide (Anandron, Nilandron): a nonsteroidal antiandrogen often used as an antineoplastic agent in treatment of prostate cancer; and
Spironolactone (Aldactone, Spiritone): a synthetic 17-spirolactone corticosteroid, which is a renal competitive aldosterone antagonist in a class of pharmaceuticals called potassium-sparing diuretics, used primarily to treat low-rennin hypertension, hypokalemia, and Conn's syndrome. These methods may employ combination compositions, which may be in combination unit dosages, or separate compositions, which may be provided separately dosed in joint packaging.

The compositions of the present invention may be administered orally, intravenously, intranasally, rectally, or by any means which delivers an effective amount of the active agent(s) to the tissue or site to be treated. Suitable dosages are those which achieve the desired endpoint. It will be appreciated that different dosages may be required for treating different disorders. An effective amount of an agent is that amount which causes a significant decrease in the targeted pathology, or progress of the pathology, or which delays the onset or reduces the likelihood of pathology in predisposed hosts. For example, the effective amount may decrease neoplastic prostate cell count, growth, or size.

Those having ordinary skill in the art will be able to empirically ascertain the most effective dose and times for administering the agents of the present invention, considering route of delivery, metabolism of the compound, and other pharmacokinetic parameters such as volume of distribution, clearance, age of the subject, etc.

The invention also provides kits specifically tailored to practicing the subject methods, including kits comprising an antiandrogenic, optionally substituted 3,3′-diindolylmethane, and an associated, such as copackaged, instructional medium describing or reciting a subject method. The recited antiandrogenic, optionally substituted 3,3′-diindolylmethane may be present in premeasured, unit dosage, and may be combined in dosage or packaging with an additional therapeutic agent, particularly a different antiandrogen.

The invention also provides business methods specifically tailored to practicing the subject methods. For example, in one embodiment, the business methods comprise selling, contracting, or licensing a subject, antiandrogenic, optionally substituted 3,3′-diindolylmethane-based method or composition.

The present invention is exemplified in terms of in vitro and in vivo activity against various neoplastic and normal cell lines. The test cell lines employed in the in vitro assays are well recognized and accepted as models for antitumor activity in animals. The mouse experimental tumor in vivo assays are also well recognized and accepted as predictive of in vivo activity in other animals such as, but not limited to, humans.

### EXEMPLARY EMPIRICAL PROTOCOLS

I. DIM is a Potent Androgen Antagonist in Human Prostate Cancer Cells.

We conducted a series of cell proliferation and gene activation studies in androgen dependent (LNCaP) and androgen independent (PC-3) human prostate cancer cell lines. LNCaP cells were derived from lymph node metastasis and PC-3 cells were derived from bone metastasis (22-25). We found that DIM is a strong antiandrogen that inhibited androgen dependent tumor cell growth and competitively inhibited androgen receptor translocation and signal transduction. In addition, DIM down regulated prostate specific antigen (PSA) expression at the transcriptional level. Results from AR competitive binding assays, nuclear translocation studies, and structural modeling computations indicate that DIM disrupts AR function in a manner similar to a chemically dissimilar synthetic antiandrogen, Casodex. Our results identify DIM as a structurally novel, naturally occurring, pure androgen antagonist having cancer preventive and therapeutic usefulness for prostate cancer. Aspects of these experiments are described in Le et al. J Biol Chem. 2003 Jun. 6;278(23):21136-45.

DIM was prepared from I3C as described (26-28) and recrystallized in toluene. The human prostate adenocarcinoma cell lines LNCaP-FGC and PC-3 were obtained from the American Type Culture Collection (Rockville, Md.). They were grown as adherent monolayers in 10% FBS-DMEM, supplemented with 4.0 g/L glucose and 3.7 g/L sodium bicarbonate in a humidified incubator at 37° C. and 5% CO2 and passaged at approximately 80% confluency. Cultures used in subsequent experiments were at less than 40 passages. Cells grown in stripped conditions were in 5% DCC-FBS-DMEM base supplemented with 4.0 g/L glucose, 3.7 g/L sodium bicarbonate, and 0.293 g/L L-Glutamine.

Cell Growth. Before the beginning of the treatments, cells were depleted of androgen for 4-7 d in medium composed of DMEM base without phenol-red and with 4.0 g/L glucose and 3.7 g/L sodium bicarbonate. During the depletion period, medium was changed every 48 h. Treatments were administered by addition of 1 μL of 1,000× solution in DMSO per mL of medium. Once the treatment period started, medium was changed daily to counter possible loss of readily metabolized compounds.

Cell Counting. Cells were harvested by trypsinization and resuspended in culture medium. Aliquots were diluted 50-fold in Isoton II (Coulter Corp., Miami, Fla.) and 200 μL duplicates were counted in a model Z1 Coulter particle counter and averaged.

[³H]Thymidine Incorporation. LNCaP cells were plated onto 24-well plates (Coming) with 2×10⁴ cells per well and treated with varying concentrations of DIM with and without 1 nM DHT for 24-48 h. [³H]Thymidine (3 μCi) were then added to each well and incubated at 37° C. for 2-3 h. Medium was removed and the cells were washed three times with 2 mL ice-cold 10% trichloroacetic acid, followed by addition of 300 mL 0.3 N NaOH to each well and then incubated at room temperature for 30 min. Aliquots (150 μL) were transferred into the scintillation vials with 4 mL of ScintiVerse BD scintillation fluid (Fisher) and counted for its radioactivity by a Beckman liquid scintillation counter.

Plasmid Reporters and Expression Vectors. The ARE responsive luciferase reporter plasmid, pPSA-630 luciferase (pPSA-Luc) was prepared as previously described (29). pPSA-Luc contains the PSA promoter region (−630 to 12) with three AREs, all of which are critical to the activity of the pPSA-Luc promoter. The MMTV-Luc contains one consensus ARE and the expression vector, pCMV-hAR constitutively expresses a fully functional human androgen receptor. The pCMV-GFP-rAR was prepared as previously described (30).

RNA Extraction, mRNA Purification and Northern Hybridization. mRNA isolation and Northern blot analyses were conducted as described previously (20, 31). PSA cDNA and the cDNA probes were biotinylated using NEBlot Phototope kit (New England Biolabs, Beverly, Mass.), purified via precipitation with 3 M sodium acetate (pH 5.2) and washed with 70% ethanol. After hybridization with cDNA probes, the membrane was incubated with streptavidin then biotinylated with alkaline phosphatase followed by the Phototope-CDP-Star assay (New England Biolabs, Beverly, Mass.), and autoradiographed. The amount of mRNA was quantified by Gel Densitometer (Bio-Rad) and normalized with β-actin as an internal control.

Analysis of Intracellular and Secreted PSA. LNCaP cells growing on 100 mm plates were treated for 24 h. Cells were lysed as previously described (20) for intracellular PSA analysis. For secreted proteins, spent medium was collected and concentrated 18-fold using Millipore Centriprep YM-10 following manufacturer's protocol (Bedford, Mass.). Protease inhibitors were added and the proteins were immunoprecipitated with 3 μg/mL monoclonal free PSA antibody (Biodesign International) for 2 h and co-immunoprecipitated overnight at 4° C. with Protein A/G agarose (Santa Cruz Biotechnology, Santa Cruz, Calif.) on a rotator. The samples were then subjected to Western blot analysis as described previously (20) using a monoclonal PSA (sc-7316) primary antibody and a goat anti-mouse-IgG-AP secondary antibody.

Transient Transfections with Reporters and Luciferase Assay. LNCaP and PC-3 cells were transfected, with some modifications, as previously described (20). For AR transactivation, cells were transfected with 0.1 μg of MMTV-Luc or pPSA-Luc per plate. Co-transfection experiments with pCMV-hAR or pCMV-GFP-rAR, 0.1 μg per plate was also used. For experiments involving GFP fluorescence imaging, treatments were not added until 30 h after transfection.

Hormone Binding Assay. LNCaP cells were grown in 5% DCC-FBS-DMEM medium supplemented with 4.0 g/L glucose and 3.7 g/L sodium bicarbonate and harvested in HBS containing 1.5 mM EDTA by scraping with a rubber policeman. The cells were placed on ice, collected by centrifugation, washed with ice-cold TKEG buffer (20 mM Tris-HCl pH 7.4, 50 mM KCl, 1 mM EDTA, 0.1 mM PMSF, and 10% glycerol) and resuspended in 250 μL/plate homogenization buffer (50 mM Tris-HCl pH 7.4, 1.5 mM EDTA, 10 mM sodium molybdate, 2.5 mM β-mercaptoethanol, 50 mM KCl, 0.1 mM PMSF, and 10% glycerol). Cells were homogenized using a Polytron apparatus at medium speed for 1 min on ice. The homogenates were centrifuged at 50,000 rpm in 4° C. for 60 min. The supernatant solution was divided into 1.0 mL aliquots, quickly frozen in a dry-ice/ethanol bath and stored at −80° C. Protein concentration was determined by the Bradford assay using bovine serum albumin as the standard. For each competitive binding assay, 5 μL of 20 nM [³H]DHT in 50% ethanol, 10 mM Tris, pH 7.5, 10% glycerol, 1 mg/mL BSA, and 1 mM DTT was placed in a 1.5 mL microcentrifuge tube. Competitive ligands were added as 1.0 μL of 100× solution in DMSO. After mixing, 95 μL of either LNCaP cell extracts or recombinant AR protein (PanVera, Madison, Wis.) was added and the solutions were vortexed and incubated at room temperature for 2-3 h. Proteins were precipitated by addition of 100 μL of 50% hydroxylapatite (HAP) slurry equilibrated in TE (50 mM Tris, pH 7.4, 1 mM EDTA) and incubated on ice for 15 min with vortexing every 5 min to resuspend the HAP. The pellet was washed with 1.0 mL ice-cold wash buffer (40 mM Tris, pH 7.4, 100 mM KCl), and centrifuged for 5 min at 10,000×g at 4° C. The supernatant was carefully aspirated and the pellet washed two more times with 1.0 mL of wash buffer. The final pellet was resuspended in 200 μL ethanol and transferred to a scintillation vial. The tube was washed with another 200 μL ethanol which was then added to the same counting vial. A negative control contained no protein and nonspecific binding was determined using 100-fold (0.1 μM) excess unlabeled DHT.

Subcellular Fractionation. Three near confluent (80-90%) cultures of LNCaP cells in 100 mm Petri dishes were used for each treatment. Treatments were added as 1 μL of 1,000× solution in DMSO per ml of medium for the indicated time. After incubation with treatments at 37° C., cytosolic and nuclear proteins were prepared as described (32,33) with modifications. Briefly, cells were lysed in hypotonic buffer (10 mM Hepes, pH 7.5) and harvested in MDH buffer (3 mM MgCl2, 1 mM DTT, 25 mM Hepes, pH 7.5). After homogenization, supernatant was saved for cytosolic proteins and nuclear proteins were extracted from the pellets using MDHK buffer (3 mM MgCl2, 1 mM DTT, 0.1 M KCl, 25 mM Hepes, pH 7.5) followed by HDK buffer (25 mM Hepes, pH 7.5, 1 mM DTT, 0.4 M KCl). Cytosolic and nuclear extracts were subsequently analyzed by Western blot analysis.

Western Blot Analysis. Following the indicated treatment, Western immunoblot analyses of androgen receptor from LNCaP cells were performed as described previously (20). In short, polyclonal AR antibodies, sc-816 and sc-815, from Santa Cruz Biotechnology, Inc. (Santa Cruz, Calif.) were used as primary antibodies with a chemiluminescence protein detection method. Blotted membranes were stained with Coomassie blue to determine protein loading or beta-actin (sc-8432, Santa Cruz Biotechnology) was used as an internal control. The amount of protein was quantified by Gel Densitometer (Bio-Rad) and normalized with P-actin when used as an internal control.

Fluorescence Imaging. PC-3 cells were plated on cover slips in six-well culture plates at 1.5×10⁵ cells per well in 5% DCC-FBS-DMEM medium. Cells were co-transfected with pCMV-GFP-rAR and pPSA-Luc or MMTV-Luc as indicated above. Cover slips were placed on microscope slides and images were taken at 1000×. Fluorescence imaging of GFP was performed using a Zeiss Axiophot 381 and Q-imaging MicroPublisher at the CNR Biological Imaging Facility of the University of California, Berkeley.

Modeling of DIM Binding to the AR Ligand Binding Domain. Quantum mechanical geometry optimizations were performed at a high level of theory, 6-31G**/MP2, for DHT, DIM, Casodex and R1881. Using these molecular coordinates, a solvent accessible surface was constructed surrounding each molecule; such a surface enables coupling of the ab initio electronic structure calculations to the solution of the Poisson-Boltzmann equation (34). The coupling was accomplished through the single and double layers of charge at the boundary and allowed for relaxation of the quantum electronic charge distribution in response to these surrounding layers. This first principles approach eliminated the need to assign fractional charges to the atoms. The induced polarization charge at the interface was then mapped onto the nodes of the elements of the solvent accessible surface. A comparison was then made between these molecules.

The atomic configuration of DHT determined experimentally, i.e., obtained from the crystal structure of the molecule in the androgen receptor, provided a template for comparison of the feasibility of the androgen receptor binding a different ligand (35). We therefore again constructed a solvent accessible surface, SR, surrounding the DHT molecule (crystal structure) and used this surface as a reference standard or template for the androgen receptor's ligand binding site. The center-of-mass of each androgen receptor ligand, DHT, Casodex, R1881 (optimized coordinates) and DIM, was translated to the center-of-mass of the template (crystal structure coordinates) and then rotated about the x, y, and z axes through the center-of-mass. We calculated the fractional surface area of the ligand, which did not fit into the binding site template as follows. For each element, i, of the ligand surface, SL, we then found its nearest neighbor element j on SR, allowing us to form the vector rij=ri-ij from element j (nearest neighbor to i of SL) on SR to element i. By forming the dot product of rij with the normal to the element at j on SR, rij*nj, we could determine whether element i of SL is inside or outside of SR. In this way, we calculated a SL, the fractional surface area of each ligand that lies outside the template DHT surface SR. This method was repeated using the crystal structure of R1881 as the binding site template (36).

DIM inhibits the proliferation and DNA synthesis of uninduced and DHT-induced LNCaP cells. The effects of DIM on human prostate cancer cell growth were examined using LNCaP and PC-3 cells. Following a 96 h treatment, DIM produced a concentration-dependent inhibition of LNCaP cell proliferation with maximal inhibition of 70% at 50 μM. At these concentrations, DIM had no observable effects on the growth of PC-3 cells. In addition, we examined the effects of varying concentrations of DIM with and without 1 nM DHT on DNA synthesis in LNCaP cells. Our results showed a concentration-dependent inhibition of DNA synthesis of these cells of up to 90% under both uninduced and androgen-induced growth conditions.

Inhibition of endogenous PSA expression by DIM. Northern blot analysis was used to examine the effect of DIM on endogenous PSA gene expression. Our data showed concentration-dependent and time-dependent decreases of up to 70% in PSA MRNA levels following DIM treatments. In addition, PSA mRNA induction by DHT with increasing time of treatment was inhibited by up to 80% by 24 h co-treatment with DIM. Furthermore, Western immunoblot analysis showed that DIM reduced levels of intracellular and secreted PSA protein to background concentrations, following DHT co-treatments. The reduction of PSA expression was comparable to the reduction in DHT-induced mRNA expression determined by Northern blot analysis. These results indicate DIM regulation of PSA expression occurring at the transcriptional level, consistent with the antiandrogenic activity of DIM observed in the cell proliferation experiments.

DIM down-regulates the activities of DHT induced reporter genes. The antiandrogenic effects of DIM were further examined with reporter assays using a MMTV-Luc promoter construct that contains one ARE and a pPSA-Luc promoter construct containing three AREs. These plasmids were transiently transfected into LNCaP cells, and by luciferase analysis, showed that DIM strongly inhibited DHT induction of androgen-responsive genes by over 50% at 1 μM, and over 90% at 10 μM in both promoter constructs. Treatment with DIM alone failed to induce transactivation of these reporter genes. These results further confirm that DIM inhibition of AR-responsive gene expression occurs at the transcriptional level.

The AR is the central modulator of DIM inhibitory effects on androgen-regulated gene expression. To confirm the importance of the AR in the transcriptional activation of the ARE promoters, we employed PC-3 cells, which exhibit little or no AR expression. We transfected these cells with the pPSA-Luc promoter and performed luciferase analysis to show that without co-transfection of an AR expression vector, DIM has no effect. In contrast, co-transfection of an AR expression vector with the pPSA-Luc reporter construct led to a concentration-dependent inhibition of DHT-induced transactivation by DIM that was similar to the effect we had observed in LNCaP cells. The same results were seen with the MMTV-Luc promoter. Moreover, DIM by itself did not induce transactivation of these reporter genes in either cell line, with or without co-transfection of the wild-type androgen receptor.

DIM competes with androgen for binding to the AR in LNCaP cells and in recombinant AR protein. Since our results implicated the AR as the focus of DIM's mode of action in prostate cells, we assessed directly the ability of DIM to bind to this receptor. Our results of competitive binding assays with both the mutant AR of LNCaP cells and a wild-type recombinant human AR demonstrate that DIM, in the micromolar concentration range, competes with labeled DHT for binding to the AR. Cyproterone acetate and Casodex, two well-known antiandrogens, were used as positive controls. DIM and Casodex exhibited similar binding affinity for the AR.

Biochemical analysis of AR cytoplasmic/nuclear distribution in cells treated with DHT and DIM. To examine the effect of DIM on nuclear translocation of the AR, both Western blot analysis and fluorescence imaging of tagged AR were conducted. LNCaP cells were treated with DIM in the presence and absence of 1 nM DHT. Cytoplasmic and nuclear protein fractions were extracted and subjected to Western blot analysis for the AR. The results show that DIM by itself had no effect on nuclear translocation and that 1 nM DHT produced a strong translocation of the AR into the nucleus. However, DHT-induced AR translocation was blocked up to 75% when cells were co-treated with DIM.

Fluorescence imaging using a pCMV-GFP-rAR co-transfected with pPSA-Luc was used to confirm and extend the results of our Western blot analyses of endogenous AR translocation. Cells treated with 1 nM DHT showed hormone-induced trafficking of the AR to be predominantly nuclear within 1 h of treatment. However, co-treatment with 50 μM DIM partially inhibited the translocation of AR induced by DHT treatment and showed distribution of the AR to be both cytoplasmic and nuclear. Furthermore, DIM treatment prevented the formation of AR foci in the nucleus. DIM alone exhibited a predominantly cytoplasmic distribution of fluorescence.

In a control experiment, the expression vector for the GFP-AR was co-transfected with the androgen-regulated reporter genes, pPSA-Luc and MMTV-Luc, to investigate the activity of the pCMV-GFP-rAR. The results verified that the activity of the chimeric receptor construct in the presence of DHT and DIM was similar to activity of the simple pCMV-AR-derived receptor. These results show that DIM both inhibits the nuclear translocation of the liganded AR and prevents the formation of nuclear AR foci.

Structural modeling of DIM binding to the AR and comparison with DHT, R1881, and Casodex. Since DIM is a strong antagonist of AR function but exhibits less than obvious structural similarity to the endogenous AR ligand, DHT, we compared the structure of DIM to DHT and other AR ligands. Results from these calculations showed similarities among DIM, DHT, the AR agonist R1881, and the AR antagonist Casodex. A comparison of the dimensions of all the ligands without their hydrogen atoms showed the experimentally determined structures of DHT and R1881 to be approximately 20 Å in the long axis, versus 18 Å for the optimized DIM and 15 Å for Casodex. All of the ligands exhibit the same width, but DIM and Casodex are twice the height of the other ligands. In addition, comparison of the crystal structure of DHT with its computationally optimized conformation showed a slight bending upward of the 3-OH end in the optimized molecule versus a more planar, slightly downward-pointing 3-OH end in the crystal structure. The same change is seen in R1881. This result indicates a slight conformational change in the ligand when it binds to the receptor's binding site.

We then compared the solvent-induced polarization charges for the AR ligands. We compared solvent-accessible surfaces for DIM, DHT, R1881 and Casodex. The results indicated a similar charge pattern and ellipsoid shapes for all of the ligands, with positive surface charge above the oxygen, fluorine or nitrogen atoms on both ends of the molecules.

Since both DIM and Casodex act as pure antiandrogens, we compared the structures of these ligands more closely. The two ligands are similar in conformation despite their considerable difference in atomic compositions. Both molecules have a planar region containing a polar atom (nitrogen for DIM, fluorine for Casodex) which can bind into the known AR binding site in a manner comparable to the 3-OH group of DHT, combined with a bulky region at the opposite end of the ligand. When rotated by 90°, to look directly down the bulky end, we observe that this end of each of the molecules tilts 30-45° relative to the distal aromatic rings, indicating a similar fit into the androgen receptor's ligand binding site. These conformations are in contrast to the more planar structures of the AR agonists, DHT and R1881.

The antiproliferative and antiandrogenic activity of DIM was observed at physiologically relevant concentrations. A man of average weight who consumes 200 g of broccoli daily will obtain approximately 12 mg of DIM. With maximum absorption of DIM, the blood concentration of DIM will reach approximately 10 μM. Therefore, in vivo concentrations of DIM from dietary Brassica vegetables represent the effective levels of DIM in vitro.

II. Activity Evaluation of DIM Derivatives in a 5-day Hershberger Assay.

Castration and testosterone capsule implantation. Rats are anesthetized with ether and then castrated. Testosterone-containing capsules are inserted subcutaneously through an incision made posterior to the scapulae. Capsules are prepared by tamping crystalline testosterone (approximately 30 mg/capsule) into silicone rubber tubing (1.6 mm id, 3.2 mm od, 25 mm length). Each end of the capsule is sealed by silicone, and wiped clean with ethanol (see, Bookstaff et al., 1990a,b; Kelce et al., 1997; Smith et al., 1977).

Hershenberger assay protocol. Operated male rats (Crj:CD(SD)IGS rats, 11 weeks of age, 6 rats/diindolylmethane/dose) are treated by gavage with a panel of 3,3′-diindolylmethane derivatives confirmed to have antiandrogenic activity in our reporter assay (supra) at 0.1, 0.5, 2.5, and 10 mg/kg/day, flutamide (10 mg/kg/day) or vehicle control. Based on optimization experiments (Yamada et al., Toxicological Sciences 5, 289-296, 2000) two capsules containing testosterone are implanted in each rat, and seven days after the operation, rats are dosed for 5 days, then euthanized on the morning of test day +6. Carboxymethylcellulose (0.5%) is used as the vehicle, and the dose volume is 5.0 ml/kg body weight. Organ weights (ventral prostate, seminal vesicles with coagulating glands, and levator ani muscle plus bulbocavernosus muscle), and serum hormone levels (testosterone and LH) are determined using commercially available RIA kits together with indices of toxicity such as body weight, food consumption, hematology, and blood biochemistry. Histopathology of the ventral prostate, seminal vesicles, and levator ani plus bulbocavernosus muscle followed standard procedure.

Castrated rats implanted with two capsules containing testosterone (6 rats/dose) are treated by gavage for 5 days with a DIM derivative (0.1, 0.5, 2.5, or 10 mg/kg/day), flutamide (10 mg/kg/day) or vehicle control. Data from body weights, food consumption, hematology, blood biochemistry, and organ weights apart from accessory sex organs, indicate that no serious systemic toxicity occurrs following the administration of the DIM derivatives or flutamide. Serum testosterone and LH levels and weights of ventral prostate and seminal vesicles in controls are similar to those of the castrated rats implanted with 2 testosterone capsules.

Our results show that DIM derivatives and flutamide dose-dependently decrease recovered mass of seminal vesicle and levator ani and bulbocavernosus muscles and ventral prostate compared with control vehicle treated animals. Similarly, histopathological analysis reveal that rats dosed with DIM derivatives or flutamide show ventral prostate and seminal vesicle atrophy, as well as atrophy of levator ani plus bulbocavemosus muscles.

III. DIM Analogs Inhibit Androgen-induced Delay of Hair Growth in the Golden Syrian Hamster.

In this study, we demonstrate that antiandrogenic DIM analogs can promote hair growth by inhibiting testosterone suppression of hair growth in the skin surrounding the flank organ of the male Syrian hamster in a protocol adapted from Mezick et a. (1999) Br J Dermatol 140 (6), 1100-1104.

Mature male golden Syrian hamsters, 101-110 g, are obtained from Charles River Laboratories (Wilmington, Mass.). Pellet implant and blood drawing procedures are performed while animals are under general anaesthesia induced by isoflurane (Aerrane®, Fort Dodge Animal Health, Overland Park, Kans.). Testosterone propionate (TP) is administered in the form of 25 mg dose time-release pellets (Inovative Research of America, Toledo, Ohio). Antiandrogenic DIM derivatives (Example II, supra) are coadministered in parallel 25 mg dose time-release pellets. Control animals receive placebo pellets, which contain only carrier/binder excipients (cholesterol, lactose, celluloses, phosphates, and stearates).

Pellets are implanted subcutaneously. A skin incision is made with scissors on the upper dorsal side of the animal, equal in length to the pellet. A horizontal pocket is created with forceps about 2 cm beyond the incision site. Pellets are placed in the pocket with forceps, and incisions are then closed with a staple suture (Autoclip®, 9 mm, Stoelting Co., Wood Dale, Ill.). Immediately after pellet implantation, the fur on and around each flank organ is shaved with electric clippers. This is designated day 0 of the study. At day 7, the fur around the flank organ is re-clipped. The amount of hair growth on the area surrounding the flank organ is visually graded at weekly intervals on a 0-3 scale (0=bald skin, 1=slight hair growth, 2 =moderate hair growth, 3=full hair growth). On day 21 the surface diameters of the left and right flank organs (encompassed within the pigmented area) are measured with vernier callipers. Animals are then killed by CO2 inhalation.

Sebaceous glands are evaluated in unstained whole mounts of ventral dermal sheets from the left ears. Each slide contains the dermal sheet peeled off from the entire ear using the separation method described by Matias and Orentreich (J Invest Dermatol 1983; 81, 43-6). The gland areas are measured using an Optomax V image analysis system (Optomax, Hollis, N.H.). The percentage change in sebaceous gland size is calculated by comparing the testosterone-treated group with the placebo-treated group. Serum testosterone is measured by radioimmunoassay at an outside laboratory (AniLytics, Inc., Gaithersburg, Md.).

Hair growth is quantified using a visual grading system (0-3 scale, where 0=no growth, 3=full hair growth). Additionally, blood is taken at various intervals during the study to assess the serum levels of testosterone in placebo- and TP-treated hamsters. Fourteen days after TP pellets are implanted, the serum testosterone is typically elevated 4.5-fold compared with placebo, and remains slightly elevated above controls after 21 and 28 days. No hair growth is observed in controls or TP-treated animals (hair growth grade=0) at day 14. By day 21 hair growth is significant in both controls and in animals co-treated with both TP and our antiandrogenic DIM analogs, whereas hair growth is insignificant in TP-treated hamsters.

We also monitor the animals for classic stimulatory effects on the flank organ and ear sebaceous glands. Our results show clearly that the 25 mg TP dose causes a marked increase in the size of the flank organ and the ear sebaceous glands after 21 days, and that this decrease is inhibited by antiandrogenic DIM analogs. Our data indicate that androgen induces well-known stimulatory responses within sebaceous structures while it suppresses hair growth in the skin around the flank organ, and that these responses and hair growth repression can be inhibited by antiandrogenic DIM analogs.

IV. Anti-androgenic Activity of DIM Derivatives in Human Skin.

Since acne formation is a multistep process accelerated by androgens, we examined whether DIM derivatives can also provide anti-androgenic activity in an established transient transfection assay in human skin fibroblasts (see, Inui et al., J Dermatological Sci, 27 (2), October 2001, 147-151. Results of these experiments clearly demonstrate that our antiandrogenic DIM derivatives can suppress androgen activity in human skin fibroblasts.

Human dermal fibroblasts are isolated from skin specimens obtained from plastic surgery operations and then maintained in Dulbecco's modified Eagle's medium supplemented with 10% fetal bovine serum.

Transfection and reporter gene assays: Human dermal fibroblasts are grown in Dulbecco's modified Eagle's medium supplemented with 10% fetal bovine serum. At 50-70% confluency in a 24-well plate, the cells are transfected using lipofectamine plusTM (Gibco-BRL) according to the manufacturer's instruction. For luciferase assays, we cotransfect 0.3 g of the reporter plasmids, mouse mammary tumor virus luciferase (MMTV-Luc, Hsiao et al., 1999, J Biol Chem 274, 22373-9) and the expression plasmids for AR (Fujimoto et al., 1999, J Biol Chem 274, 8316-21) and pSG5-AR (Hsiao et al., supra). The pRL-CMV vector, the Renilla luciferase control reporter vector driven by the CMV immediate-early enhancer/promoter, is co-transfected as an internal control. At 24 h after transfection, we put fresh medium with methyltrienolone (R1881, a synthetic androgen) with and without our tested antiandrogenic DIM derivatives (Example II, supra) at concentrations of 1, 10, 100 and 1000 ug/ml. At 48 h after transfection, the cells are harvested for luciferase assays. Luciferase activities are measured by luminometer using the Dual-LuciferaseTM reporter assay system (Promega).

| | |
|---|---|
| 1. | Wingo, et al. (1997) CA Cancer J Clin 47, 239-242 |
| 2. | Small, E. J. (1998) Drugs Aging 13, 71-81. |
| 3. | Shimizu, et al. (1991) Br J Cancer 63, 963-966. |
| 4. | Haenszel, W., and Kurihara, M. (1968) J Natl Cancer Inst 40, 43-68. |
| 5. | Denis, L., Morton, M. S., and Griffiths, K. (1999) Eur Urol 35, |
| | 377-387. |
| 6. | Kolonel, et al. (2000) Cancer Epidemiol Biomarkers Prev 9, |
| | 795-804. |
| 7. | Terry, P., Wolk, A., Persson, I., and Magnusson, C. (2001) Jama |
| | 285, 2975-2977. |
| 8. | Terry, P., Vainio, H., Wolk, A., and Weiderpass, E. (2002) Nutr |
| | Cancer 42, 25-32 |
| 9. | Voorrips, et al. (2000) Am J Epidemiol 152, 1081-1092. |
| 10. | Kristal, A. R., and Lampe, J. W. (2002) Nutr Cancer 42, 1-9 |
| 11. | Bradlow, et al. (1991) Carcinogenesis 12, 1571-1574. |
| 12. | Grubbs, et al. (1995) Anticancer Res 15, 709716. |
| 13. | Chen, et al. (1998) Carcinogenesis 19, 16311639. |
| 14. | Wattenberg, L. W., and Loub, W. D. (1978) Cancer Res 38, |
| | 1410-1413. |
| 15. | Wattenberg, L. W. (1980) J Environ Pathol Toxicol 3, 35-52. |
| 16. | Cover, et al. (1998) J Biol Chem 273, 3838-3847. |
| 17. | Ge, X., Fares, F. A., and Yannai, S. (1999) Anticancer Res 19, |
| | 3199-3203. |
| 18. | Chinni, et al. (2001) Oncogene 20, 2927-2936. |
| 19. | Chinni, S. R., and Sarkar, F. H. (2002) Clin Cancer Res 8, |
| | 1228-1236. |
| 20. | Hong, et al. (2002) Carcinogenesis 23, 1297-1305. |
| 21. | Ge, et al. (1996) Biochem Biophys Res Commun 228, 153-158. |
| 22. | Veldscholte, et al. (1992) J Steroid Biochem Mol Biol 41, 665-669. |
| 23. | Webber, M. M., Bello, D., and Quader, S. (1997) Prostate 30, 58-64. |
| 24. | Wang, M., and Stearns, M. E. (1991) Differentiation 48, 115-125. |
| 25. | Kaighn, et al. (1979) Invest Urol 17, 16-23. |
| 26. | Bradfield and Bjeldanes (1987) J Toxicol Environ Health 21, 311323 |
| 27. | Grose, K. R., and Bjeldanes, L. F. (1992) Chem Res Toxicol 5, |
| | 188-193. |
| 28. | Bjeldanes, et al. (1991) Proc Natl Acad Sci USA 88, 9543-9547. |
| 29. | Sato, et al. (1997) J Biol Chem 272, 17485-17494. |
| 30. | Roy, et al. (2001) Ann N Y Acad Sci 949, 44-57. |
| 31. | Riby, et al. (2000) Biochemistry 39, 910-918. |
| 32. | Hong, et al. (2002) Biochem Pharmacol 63, 1085-1097. |
| 33. | Riby, et al. (2000) Biochem Pharmacol 60, 167-177. |
| 34. | Wilson, et al. (1997) Chem. Phys. Lett. 267, 431-437 |
| 35. | Sack, et al. (2001) Proc Natl Acad Sci USA 98, 4904-4909 |
| 36. | Matias, et al. (2000) J Biol Chem 275, 26164-26171 |

The foregoing descriptions of particular embodiments and examples are offered by way of illustration and not by way of limitation. All publications and patent applications cited in this specification and all references cited therein are herein incorporated by reference as if each individual publication or patent application or reference were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of providing an antiandrogen to a host determined to be in need thereof, the method comprising the steps of:
contacting the host with an effective amount of an antiandrogenic, optionally substituted 3,3′-diindolylmethane (DIM); and
detecting a resultant antiandrogenic response in the host.

2. The method of claim 1 wherein the method further comprises, prior to the contacting step, determining that the host is in need of the antiandrogen.

3. The method of claim 1 wherein the host is a human patient determined to be subject or predisposed to an androgen-dependent pathology, and the resultant antiandrogenic response is a reduction in the pathology or progress of the pathology.

4. The method of claim 1 wherein the host is a human patient determined to be subject or predisposed to an androgen-dependent pathology selected from the group consisting of prostate hyperplasia, acne, androgenetic alopecia and hirsutism, and the resultant antiandrogenic response is a reduction in the pathology or progress of the pathology.

5. The method of claim 4 wherein the patient is determined to be subject to prostate cancer.

6. The method of claim 4 wherein the patient is determined to be predisposed to prostate cancer.

7. The method of claim 1 wherein the optionally substituted 3,3′-diindolylmethane has the formula: 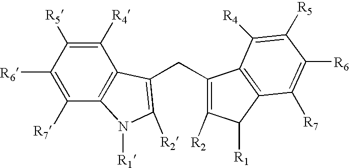
where R.sub.1, R.sub.2, R.sub.4, R.sub.5, R.sub.6, R.sub.7, R.sub.1′, R.sub.2′, R.sub.4′, R.sub.5′, R.sub.6′ and R.sub.7′ individually and independently, are hydrogen or a substituent selected from the group consisting of a halogen, a hydroxyl, a linear or branched alkyl or alkoxy group of one to ten carbons, and a nitro group.

8. The method of claim 7 wherein R.sub.1, R.sub.2, R.sub.4, R.sub.5, R.sub.6, R.sub.7, R.sub.1′, R.sub.240 , R.sub.4′, R.sub.5′, R.sub.6′ and R.sub.7′ include a substituent selected from the group consisting of a halogen, a hydroxyl, a linear or branched alkyl or alkoxy group of one to ten carbons, and a nitro group.

9. The method of claim 8 wherein the linear or branched alkyl or alkoxy group is one to five carbons.

10. The method of claim 8 wherein the halogen is selected from the group consisting of chlorine, bromine and fluorine.

11. The method of claim 8, wherein R.sub.1, R.sub.2, R.sub.4, R.sub.6, R.sub.7, R.sub.1′, R.sub.2′, R.sub.4′, R.sub.6′, and R.sub.7′ are hydrogen, and R.sub.5 and R.sub.5′ are a halogen.

12. The method of claim 8, wherein R.sub.2, R.sub.4, R.sub.5, R.sub.6, R.sub.7, R.sub. 2′, R.sub.4′, R.sub.5′, R.sub.6′, and R.sub.7′ are hydrogen, and R.sub.1 and R.sub.1′ are an alkyl or alkoxyl having from one to ten carbons.

13. The method of claim 8, wherein R.sub.1, R.sub.4, R.sub.5, R.sub.6, R.sub.7, R.sub.1′, R.sub.4′, R.sub.5′, R.sub.6′, and R.sub.7′ are hydrogen, and R.sub.2 and R.sub.2′ are an alkyl of one to ten carbons.

14. The method of claim 8, wherein R.sub.1, R.sub.2, R.sub.4, R.sub.6, R.sub.7, R.sub.1′, R.sub.2′, R.sub.4′, R.sub.6′, and R.sub.7′ are hydrogen, and R.sub.5 and R.sub.5′ are nitro.

15. The method of claim 1 wherein the optionally substituted 3,3′-diindolylmethane is 3,3′-diindolylmethane.

16. The method of claim 3 wherein the optionally substituted 3,3′-diindolylmethane is 3,3′-diindolylmethane.

17. The method of claim 4 wherein the optionally substituted 3,3′-diindolylmethane is 3,3′-diindolylmethane.

18. The method of claim 5 wherein the optionally substituted 3,3′-diindolylmethane is 3,3′-diindolylmethane.

19. The method of claim 6 wherein the optionally substituted 3,3′-diindolylmethane is 3,3′-diindolylmethane.

20. The method of claim 4 wherein the optionally substituted 3,3′-diindolylmethane is perfluoro-3,3′-diindolylmethane.

21. The method of claim 5 wherein the optionally substituted 3,3′-diindolylmethane is perfluoro-3,3′-diindolylmethane.

22. The method of claim 6 wherein the optionally substituted 3,3′-diindolylmethane is perfluoro-3,3′-diindolylmethane.
